(19) United States
(12) Patent Application Publication
(10) Pub. No.: US 2018/0271703 A1
(43) Pub. Date: Sep. 27, 2018

(54) SURGICAL GLOVES OR FINGERTIP COVERS WITH SENSORS FOR INSTRUMENT CONTROL

(71) Applicant: Novartis AG, Basel (CH)

(72) Inventor: Paul R. Hallen, Colleyville, TX (US)

(21) Appl. No.: 15/913,242

(22) Filed: Mar. 6, 2018

Related U.S. Application Data
(60) Provisional application No. 62/474,688, filed on Mar. 22, 2017.

(51) Int. Cl. A61F 9/007; A61B 5/11; A61F 9/008; A61B 5/00; A61B 3/00

(52) U.S. Cl. CPC A61F 9/00745; A61B 5/11; A61F 9/00821; A61B 2017/00207; A61B 3/0016; A61B 2562/0247; A61B 5/6806

(57) ABSTRACT

Systems and methods are provided for fingertip control of an ophthalmic surgical instrument during ophthalmic surgery. A hand control unit worn by the ophthalmic surgeon comprises at least one fingertip cover, with at least one sensor in the fingertip cover. The sensor may detect the surgeon's selection of the ophthalmic surgical instrument, the amount of finger pressure applied by the surgeon, and other parameters, allowing the surgeon to use fingertip action to control a function of the ophthalmic surgical instrument.

### FIELD OF THE INVENTION

This disclosure relates generally to ophthalmic surgery, and in particular to systems and methods for improvements in ophthalmic surgery.

### BACKGROUND OF THE INVENTION

A number of different types of ophthalmic surgeries are known and performed to treat various eye conditions. Such surgeries typically involve use by the eye surgeon of one or more hand-held instruments for performing the desired procedure.

For example, in certain vitreoretinal procedures, the surgeon may introduce an illumination probe into the posterior segment in order to see the area, a cannula for irrigation, and a vitrectomy probe having cutting and vacuum functions. As another example, in certain cataract surgeries, the surgeon may use a phacoemulsification instrument capable of delivering ultrasound for phacoemulsification of the cataractous lens. As a further example, a surgeon may use an ophthalmic laser for photocoagulation or to perform a trabeculectomy for treating glaucoma.

During current ophthalmic surgeries, while the surgeons use the instruments to perform the procedures, the surgeons often do not operate the controls for the functionality of the instruments, such as turning them on or off or adjusting a parameter such as the amount of energy delivered, irrigation delivered, or suction applied. This is largely due to the fact that the surgeon's hands are occupied with manipulating the instruments as well as the fact that the surgeon's hands need to remain sterile during the procedure, whereas the electronic control systems for controlling the functionality of the instruments are typically non-sterile. Accordingly, the electronic control systems for controlling the functionality of the instruments are often controlled by someone other than the surgeon, such as a nurse interacting with the systems. In certain systems, the surgeon may use multi-switch foot pedals to control the electronic systems that control the functionality of the instruments.

In either case, whether the instrument functionality is controlled by someone other than the surgeon or controlled by the surgeon using foot pedals, the instrument control is sub-optimal in terms of input efficiency, accuracy, safety, and/or convenience.

### SUMMARY OF THE INVENTION

The inventions described herein provide systems and methods for improvements in ophthalmic surgery.

In some embodiments a system is provided for control of an ophthalmic surgical instrument during ophthalmic surgery. The system comprises an electronic console, which may comprise a microprocessor, a memory device, and a connection port. An ophthalmic surgical instrument may be connected to the connection port of the electronic console. The system further comprises a hand control unit comprising at least one fingertip cover, at least one sensor in the fingertip cover, and a transmitter for transmitting a signal from the sensor of the hand control unit. The signal from the hand control unit may be received by an input receiver located in, for example, the electronic console or the ophthalmic surgical instrument. The signal from the hand control unit controls a function of the ophthalmic surgical instrument. The hand control unit may be a surgical glove, with the sensor(s) embedded in the fingertip cover(s) that form part of the surgical glove.

The function of the ophthalmic surgical instrument that may be controlled based on the signal from the hand control unit may include one or more of the following: turning the instrument on, turning the instrument off, selecting a mode of operation of the instrument, and/or adjusting a parameter of the instrument.

In one example, the ophthalmic surgical instrument comprises an ultrasonic probe. The signal from the hand control unit may be used, for example, to control an amount of ultrasound energy delivered from the ultrasonic probe.

In another example, the ophthalmic surgical instrument comprises a vitrectomy probe. The signal from the hand control unit may be used, for example, to control a cut rate of the vitrectomy probe, and/or to control an amount of suction applied through a vacuum port of the vitrectomy probe.

In another example, the ophthalmic surgical instrument comprises an aspiration cannula. The signal from the hand control unit may be used, for example, to control an amount of suction applied through the aspiration cannula.

In another example, the ophthalmic surgical instrument comprises an infusion cannula. The signal from the hand control unit may be used, for example, to control an amount of fluid delivered through the infusion cannula.

In another example, the ophthalmic surgical instrument comprises an intraocular lens injector. The signal from the hand control unit may be used, for example, to cause an intraocular lens to be delivered from the intraocular lens injector.

In another example, the ophthalmic surgical instrument comprises a diathermy instrument. The signal from the hand control unit may be used, for example, to control an amount of energy delivered from the diathermy instrument.

In another example, the ophthalmic surgical instrument comprises an ophthalmic laser. The signal from the hand control unit may be used, for example, to control an amount of laser treatment power. Additionally or alternatively, the signal from the hand control unit may be used to select a mode of operation, such as causing the laser to emit a continuous beam, a single burst, or a series of pulses.

In another example, the ophthalmic surgical instrument comprises scissors. The signal from the hand control unit may be used, for example, to control one or more of a cutting rate and a cutting pressure of the scissors.

In another example, the ophthalmic surgical instrument comprises forceps. The signal from the hand control unit may be used, for example, to control one or more of a closing rate and a holding pressure of the forceps.

Various types of sensors may be used for the sensor(s) in the fingertip cover(s) of the hand control unit. For example, the hand control unit may have one or more pressure sensors, motion sensors, accelerometer sensors, position sensors, relative proximity sensors, RFID identification sensors, or other suitable sensors.

In some embodiments, the system further may comprise a display and a headset, wherein the system is configured so that a user wearing the headset can point to a target location on the display at least in part by gazing at the target location on the display. Pointing to the target location in this way while activating a sensor in the hand control unit to “click” the target location can select the desired functionality.

In some embodiments, a hand control unit is provided for use in a system as described above for control of an ophthalmic surgical instrument during ophthalmic surgery. The hand control unit may comprise at least one fingertip cover, at least one sensor in the fingertip cover, and a transmitter for transmitting a signal from the sensor of the hand control unit to an input receiver for controlling a function of the ophthalmic surgical instrument.

In further embodiments, a method for controlling an ophthalmic surgical instrument during ophthalmic surgery is provided. The method comprises transmitting a signal from a hand control unit, receiving the signal at an input receiver, and based on the signal from the hand control unit, controlling a function of an ophthalmic surgical instrument. The method may be implemented using a system as described herein.

### BRIEF DESCRIPTION OF THE DRAWINGS

FIG. 1 shows an example of a system for control of an ophthalmic surgical instrument.

FIG. 2 shows a detailed view of the hand control unit of the system of FIG. 1.

FIG. 3 shows another example of a hand control unit.

### DETAILED DESCRIPTION

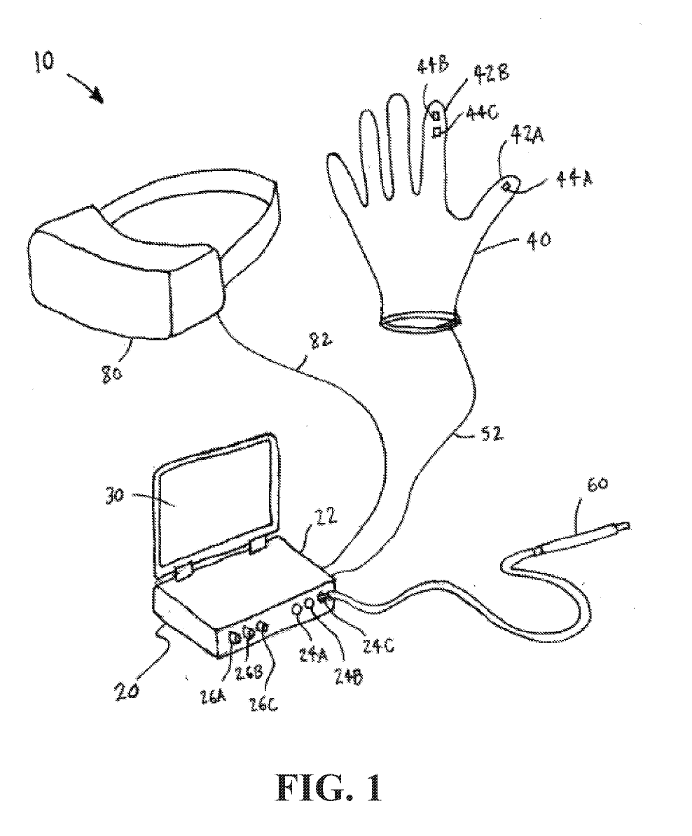

FIG. 1 shows an example of a system for control of an ophthalmic surgical instrument during ophthalmic surgery. The system 10 comprises an electronic console 20 comprising a housing 22 in which may be contained one or more microprocessors (not shown) and one or more memory devices (not shown). In many respects, the electronic console 20 may be similar to existing electronic consoles, such as the electronic console of the CONSTELLATION® Vision System from Alcon Laboratories, Inc.

In this example, the housing 22 has a series of connection ports 24A, 24B, 24C to which ophthalmic surgical instruments may be connected. The housing 22 also has a series of control mechanisms 26A, 26B, 26C, which may be any form for allowing operator control, such as knobs, dials, switches, and the like. Such input control mechanisms may additionally or alternatively be on a remote control unit (not shown) that communicates with the electronic console 20 via remote control. In this example, the electronic console 20 also includes a display 30.

The system 10 further comprises a hand control unit 40. In this illustrated example, the hand control unit 40 is in the form of a surgical glove, for covering the entire hand. Accordingly, this example has five fingertip covers, two of which are numbered as fingertip covers 42A, 42B. The hand control unit 40 may be generally manufactured of a polymer, such as a standard polymer suitable in surgical procedures. It may be worn by the surgeon in place of standard surgical gloves, or outside of or inside of standard surgical gloves.

At least one of the fingertip covers has one or more sensors on it or embedded in it. In this example fingertip cover 42A has sensor 44A embedded in it, and fingertip cover 42B has sensors 44B and 44C embedded in it.

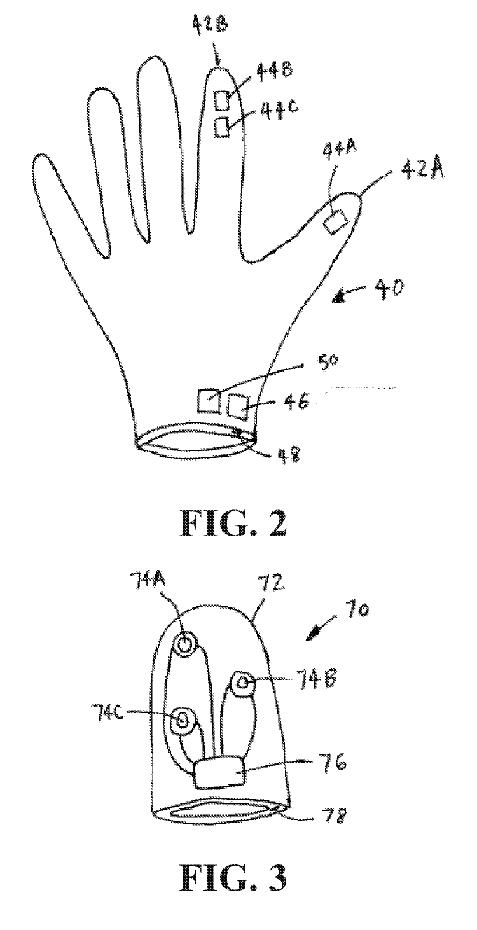

FIG. 2 shows a detailed view of the hand control unit 40 of FIG. 1. The hand control unit 40 may have a power supply such as a battery 46 and a port 48 for attaching a power cord for charging the battery 46. The hand control unit 40 also has a transmitter 50 for transmitting signals from the sensors 44A, 44B, 44C to an input receiver that is located in another system component, such as the electronic console or the ophthalmic surgical instrument itself. The transmitter 50 may transmit the signals wirelessly by various wireless transmissions as are known in the art. Alternatively, the transmitter 50 may send signals over a wire that connects the hand control unit 40 to the electronic console 20. The connecting wire may be connected through port 48 or in another suitable manner.

Various types of sensors may be used for the sensors 44A, 44B, 44C in the fingertip covers 42A, 42B of the hand control unit 40. For example, one or more of the sensors 44A, 44B, 44C may be a pressure sensor, motion sensor, accelerometer sensor, position sensor, relative proximity sensor, RFID identification sensor, or another suitable sensor.

As shown in FIG. 1, an ophthalmic surgical instrument 60 may be connected to one of the connection ports 24A, 24B, 24C of the electronic console 20. A number of ophthalmic surgical instruments may be used, for performing both anterior segment and posterior segment procedures, including, for example, vitrectomy procedures, cataract surgery, glaucoma surgery, and many other procedures known in the art.

The system 10 includes an input receiver (not shown) for receiving the signals from the transmitter 50 of the hand control unit 40. The input receiver may be located, for example, in the electronic console 20 or in the ophthalmic surgical instrument 60. In FIG. 1, line 52 schematically shows the signal connection between hand control unit 40 and electronic console 20; however, the signal connection may be between hand control unit 40 and ophthalmic surgical instrument 60. As mentioned above, this may be a wireless connection, such that there is no actual wire or other physical connection between the two devices.

In accordance with the invention, the signal from the hand control unit 40 controls a function of the ophthalmic surgical instrument 60. The function of the ophthalmic surgical instrument 60 that may be controlled based on the signal from the hand control unit may include one or more of the following: turning the instrument on, turning the instrument off, selecting a mode of operation of the instrument, and/or adjusting a parameter of the instrument.

In one example, the ophthalmic surgical instrument 60 comprises an ultrasonic probe. The ultrasonic probe may be, for example, a phacoemulsification ultrasonic probe as is used in cataract surgery. The signal from the hand control unit 40 may be used, for example, to turn the ultrasound on, to turn the ultrasound off, and/or to control an amount of ultrasound energy delivered from the ultrasonic probe.

In another example, the ophthalmic surgical instrument 60 comprises a vitrectomy probe. The signal from the hand control unit may be used, for example, to turn the cutting action on, to turn the cutting action off, to control a cut rate of the vitrectomy probe, to turn vacuum suction on, to turn vacuum suction off, and/or to control an amount or flow rate of suction applied through a vacuum port of the vitrectomy probe.

In another example, the ophthalmic surgical instrument 60 comprises an aspiration, or suction or vacuum, cannula. The signal from the hand control unit may be used, for example, to turn suction on, to turn suction off, and/or to control an amount or flow rate of suction applied through the aspiration cannula.

In another example, the ophthalmic surgical instrument 60 comprises an infusion, or irrigation, cannula. The signal from the hand control unit may be used, for example, to turn fluid delivery on, to turn fluid delivery off, and/or to control an amount or flow rate of fluid delivered through the infusion cannula.

In another example, the ophthalmic surgical instrument 60 comprises an intraocular lens injector. The signal from the hand control unit may be used, for example, to cause an intraocular lens to be delivered from the intraocular lens injector.

In another example, the ophthalmic surgical instrument 60 comprises a diathermy instrument. Diathermy may be used, for example, for coagulation to stop bleeding. The signal from the hand control unit may be used, for example, to turn the diathermy instrument on, to turn the diathermy instrument off, and/or to control an amount of energy delivered from the diathermy instrument.

In another example, the ophthalmic surgical instrument 60 comprises an ophthalmic laser. Ophthalmic lasers are used in various ophthalmic procedures, for functions such as tissue cutting, photocoagulation, and/or fragmentation. The laser may be selected from any suitable type of laser used for ophthalmic procedures, including for example excimer lasers, Nd:YAG lasers, and femtosecond lasers. The signal from the hand control unit may be used, for example, to turn the laser on, to turn the laser off, and/or to control an amount of laser treatment power delivered. Additionally or alternatively, the signal from the hand control unit may be used to select a mode of operation, such as causing the laser to emit a continuous beam, a single burst, or a series of pulses.

In another example, the ophthalmic surgical instrument 60 comprises scissors. The signal from the hand control unit may be used, for example, to close the scissors, to open the scissors, and/or to control one or more of a cutting rate and a cutting pressure of the scissors.

In another example, the ophthalmic surgical instrument 60 comprises forceps. The signal from the hand control unit may be used, for example, to close the forceps, to open the forceps, and/or to control one or more of a closing rate and a holding pressure of the forceps.

In another example, the ophthalmic surgical instrument 60 comprises an illumination probe. Illumination probes are commonly used, for example, in vitreoretinal procedures, for illuminating the posterior segment. The signal from the hand control unit may be used, for example, to turn the light on, to turn the light off, and/or to adjust the amount of light delivered from the illumination probe.

FIG. 3 shows an example of another hand control unit 70. In this example, the hand control unit 70 comprises a single fingertip cover 72. The hand control unit 70 may be generally manufactured of a polymer, such as a standard polymer suitable in surgical procedures. It may be worn by the surgeon outside of or inside of standard surgical gloves.

The fingertip cover 72 has one or more sensors on it or embedded in it. In this example fingertip cover 72 has sensors 74A, 74B, and 74C embedded in it. The sensors 74A, 74B, 74C may be similar to sensors 44A, 44B, 44C. The hand control unit 70 may have a unit 76 which acts as a combined power supply and transmitter and a port 78 for attaching a power cord for charging. The transmitter of the unit 76 transmits signals from the sensors 74A, 74B, 74C to an input receiver that is located in another system component, such as the electronic console or the ophthalmic surgical instrument itself. The transmitter of the unit 76 may transmit the signals wirelessly by various wireless transmissions as are known in the art. Alternatively, the transmitter may send signals over a wire that connects the hand control unit 70 to the electronic console 20. The connecting wire may be connected through port 78 or in another suitable manner.

The system 10 of FIG. 1 includes an optional headset 80 that the surgeon may use for certain vision-control or gaze-control functionality. In FIG. 1, line 82 schematically shows the signal connection between headset 80 and electronic console 20; however, this may be a wireless connection, such that there is no actual wire or other physical connection between the two devices.

In use, a surgeon wearing the headset 80 can point to a target location on the display 30 by gazing at the target location on the display 30. The target location may be a digital control object, such as a graphical button, arrow, value, mode, submode, or diagnostic image, etc., on a 2D or 3D display. Pointing to the target location in this way while activating a sensor in the hand control unit to “click” the target location can select, activate, or control the desired functionality.

The system of FIG. 1 may be used for various advantages in ophthalmic surgery. The physician wears the hand control unit 40 or 70 during the surgical procedure. The physician then manipulates the ophthalmic surgical instrument(s) 60 to perform the various steps of the procedure. In doing so, the sensor(s) of the hand control unit may be activated in various ways.

In one example, ophthalmic surgical instrument 60 has an identification chip in its handle. When the surgeon grasps the handle with the hand control unit 40 or 70, a sensor in the hand control unit, such as an RFID identification sensor, identifies the ophthalmic surgical instrument that has been grasped by sensing its identification chip. This signal from the sensor in the hand control unit may then be used to turn on the functionality of that particular ophthalmic surgical instrument. Similarly, when the surgeon releases that ophthalmic surgical instrument, the sensor in the hand control unit recognizes the release, by no longer sensing the chip in the instrument. This signal may then be used to turn off the functionality of the instrument. Thus, the sensor identifies when the surgeon has selected or deselected an instrument, by gripping it or releasing it, and the instrument is accordingly activated or deactivated.

In another example, the hand control unit 40 or 70 may include a pressure sensor. When the surgeon increases pressure of the grip on the handle of an ophthalmic surgical instrument 60, the sensor detects that increase in pressure, which signal may then control a parameter of functionality of the instrument.

For example, the physician may increase grip pressure to increase ultrasound energy, laser energy, vacuum or suction force, or infusion or irrigation flow rate. Similarly, the physician may decrease grip pressure to decrease each of these parameters. Relative pressure may also be used to select a surgical mode, start a function, stop a function, and/or increase or decrease other device parameter functionality.

For example, a surgeon may grip a vitrectomy probe, at which time a sensor in the hand control unit identifies a chip or sensor in the vitrectomy probe. With increased fingertip pressure, a mode is activated that initiates cutting at a preset value. Then, as fingertip pressure is increased or decreased, vacuum suction is correspondingly linearly increased or decreased with fingertip pressure. Release of fingertip pressure immediately stops the cutting mode.

As another example, a surgeon may grip an ultrasonic probe, at which time a sensor in the hand control unit identifies a chip or sensor in the ultrasonic probe. With increased fingertip pressure, a mode is activated that initiates delivery of ultrasound energy. Then, as fingertip pressure is increased or decreased, the amount of ultrasound energy delivered is linearly increased or decreased with fingertip pressure. Release of fingertip pressure immediately stops the ultrasound delivery.

As another example, a surgeon may grip a laser, at which time a sensor in the hand control unit identifies a chip or sensor in the laser. A single finger press can deliver a laser pulse. A double finger press, like a double-click on a computer mouse, can activate a pulsed laser mode. Continuous finger pressure can keep the laser on as a continuous beam.

A hand control unit as described herein may be sterile and within the sterile field during ophthalmic surgery. In the case of a hand control unit that is in the form of a glove, the hand control unit itself may serve as the surgical glove. Alternatively, a hand control unit as described herein may be worn underneath or on top of standard surgical gloves.

As can be appreciated, a hand control unit as described herein enables the surgeon to control functionality of the instruments with direct finger control. This provides the ability to have sterile controls of systems in a rapid, accurate, precise, automatic, safer, and natural fashion. Direct control by the surgeon is much faster and certain then communicating needs to an assistant verbally. The response time is much faster than foot pedal activation and deactivation, particularly important during critical ophthalmic surgical maneuvers, such as near the lens capsule or retina.

The systems and methods as described herein may be used in conjunction with other sensor-based functionality. For example, fingertip sensors interacting with surgical device sensors and resident sensors in entry cannulas can precisely determine a three-dimensional location of an instrument tip within the eye to ensure safety. For example, if an instrument is too close to a natural crystalline lens, retina, or capsule, a warning or automatic safeguard can be activated.

It will be appreciated that the systems and methods as described herein provide advancements and advantages. The above embodiments are meant as illustrative examples only. Other embodiments are possible within the scope of the disclosure and the appended claims.

What is claimed is:

1. A system for control of an ophthalmic surgical instrument during ophthalmic surgery, comprising:
an electronic console comprising a connection port;
an ophthalmic surgical instrument connected to the connection port of the electronic console;
a hand control unit comprising at least one fingertip cover, at least one sensor in the fingertip cover, and a transmitter for transmitting a signal from the sensor of the hand control unit; and
an input receiver for receiving the signal transmitted from the hand control unit;
wherein the signal from the hand control unit controls a function of the ophthalmic surgical instrument.

2. A system as recited in claim 1, wherein the hand control unit comprises a surgical glove.

3. A system as recited in claim 1, wherein the ophthalmic surgical instrument comprises an ultrasonic probe, and wherein the signal from the hand control unit controls an amount of ultrasound energy delivered from the ultrasonic probe.

4. A system as recited in claim 1, wherein the ophthalmic surgical instrument comprises a vitrectomy probe, and wherein the signal from the hand control unit controls a cut rate of the vitrectomy probe.

5. A system as recited in claim 1, wherein the ophthalmic surgical instrument comprises an aspiration cannula, and wherein the signal from the hand control unit controls an amount of suction applied through the aspiration cannula.

6. A system as recited in claim 1, wherein the ophthalmic surgical instrument comprises an infusion cannula, and wherein the signal from the hand control unit controls an amount of fluid delivered through the infusion cannula.

7. A system as recited in claim 1, wherein the ophthalmic surgical instrument comprises an intraocular lens injector, and wherein the signal from the hand control unit causes an intraocular lens to be delivered from the intraocular lens injector.

8. A system as recited in claim 1, wherein the ophthalmic surgical instrument comprises a diathermy instrument, and wherein the signal from the hand control unit controls an amount of energy delivered from the diathermy instrument.

9. A system as recited in claim 1, wherein the ophthalmic surgical instrument comprises an ophthalmic laser.

10. A system as recited in claim 9, wherein the signal from the hand control unit controls an amount laser treatment power.

11. A system as recited in claim 9, wherein the signal from the hand control unit causes the laser to emit a continuous beam.

12. A system as recited in claim 9, wherein the signal from the hand control unit causes the laser to emit a single burst.

13. A system as recited in claim 9, wherein the signal from the hand control unit causes the laser to emit a series of pulses.

14. A system as recited in claim 1, wherein the ophthalmic surgical instrument comprises scissors, and wherein the signal from the hand control unit controls one or more of a cutting rate and a cutting pressure of the scissors.

15. A system as recited in claim 1, wherein the ophthalmic surgical instrument comprises forceps, and wherein the signal from the hand control unit controls one or more of a closing rate and a holding pressure of the forceps.

16. A system as recited in claim 1, wherein the at least one sensor of the hand control unit comprises a pressure sensor.

17. A system as recited in claim 1, wherein the at least one sensor of the hand control unit comprises a motion sensor.

18. A system as recited in claim 1, wherein the system further comprises a display and a headset, wherein the system is configured so that a user wearing the headset can select a target location on the display at least in part by gazing at the target location on the display.

19. A hand control unit for use in a system for control of an ophthalmic surgical instrument during ophthalmic surgery, the hand control unit comprising:
at least one fingertip cover;
at least one sensor in the fingertip cover; and
a transmitter for transmitting a signal from the sensor of the hand control unit to control a function of the ophthalmic surgical instrument.

20. A method for controlling an ophthalmic surgical instrument during ophthalmic surgery, comprising:
transmitting a signal from a hand control unit;
receiving the signal at an input receiver;
based on the signal from the hand control unit, controlling a function of an ophthalmic surgical instrument.
